(19) United States
(12) Patent Application Publication
(10) Pub. No.: US 2026/0193156 A1
(43) Pub. Date: Jul. 9, 2026

(54) ENERGY-EFFICIENT PROCESS FOR SEPARATING BUTENES FROM C4-HYROCARBONS STREAMS

(71) Applicant: EVONIK OXENO GMBH & CO. KG, Marl (DE)

(72) Inventors: Niklas PAUL, Marl (DE); Armin Matthias RIX, Marl (DE); Moritz SCHRÖDER, Münster (DE); Philip LUTZE, Dinslaken (DE); Martina HEITZIG, Dinslaken (DE); Claudia WALLERT, Haltern am See (DE); Tanita Valèrie SIX, Dortmund (DE); Andreas OLDENKOTT, Haltern am See (DE); Benjamin WOLDT; Martin WÜLLER, Marl (DE)

(73) Assignee: EVONIK OXENO GMBH & CO. KG, Marl (DE)

(21) Appl. No.: 19/131,676

(22) PCT Filed: Nov. 24, 2023
(86) PCT No.: PCT/EP2023/082999

§ 371 Date: May 21, 2025

(30) Foreign Application Priority Data
Nov. 30, 2022 (EP) 22210409.3

(51) Int. Cl. C07C 7/08; B01D 3/00; B01D 3/14; B01D 3/40

(52) U.S. Cl. CPC C07C 7/08; B01D 3/007; B01D 3/148; B01D 3/40

(57) ABSTRACT

The invention relates to a process for separation of butenes from C4-hydrocarbon streams containing not only the butenes but also butanes by extractive distillation with a suitable solvent. The process according to the invention features heat integration with which the heat of the solvent is utilized for heating and/or at least partially evaporating different streams and the use of a heat pump for electrification of the process.

The present invention relates to a process for separation of butenes from C4-hydrocarbon streams containing not only the butenes but also butanes by extractive distillation with a suitable solvent. The process according to the invention features heat integration with which the heat of the solvent is utilized for heating and/or at least partially evaporating different streams and the use of a heat pump for electrification of the process.

The separation of butane-butene mixtures by extractive distillation is known per se. This employs an aprotic solvent (for example N-methyl-2-pyrrolidone (NMP) or acetonitrile (ACN)) to increase the relative volatility of the alkanes compared to the alkenes. In one extractive distillation column, the absorber, the butenes are preferably dissolved in the solvent and the butanes are separated as tops product. The laden solvent is subsequently freed of the butenes in a stripping column, the desorber, at elevated temperature and/or reduced pressure, and said butenes are obtained in enriched form as tops product. The solvent freed of butenes is then recycled to the extractive distillation.

Heat integration is of great importance to the economy of the process due to the high solvent to feed ratio. Hot solvent is obtained at the bottom of the desorber and the energy content thereof may be utilized in various ways. There are also ever-increasing societal and political demands to save CO₂. This too has an ever more marked effect on the economy of the process. US 2014/0124358 A1 presents a process for selective extraction of olefins which is said to solve the problem of heat integration. This document proposes utilizing the energy content of the hot solvent for heating a sidestream from the desorber, for heating the bottoms product of the absorber which is passed to the desorber, for heating one or more sidestreams of the absorber and for preheating the feed stream.

However, the solution proposed in the prior art can solve the problem of ideally full recovery of the energy streams present in the system only incompletely or via relatively complex constructions. In addition, the solution proposed therein can only make a small contribution, if any, to reducing the CO₂ emission.

The problem addressed by the present invention is accordingly that of providing a process where an improved, ideally maximum possible, energy recovery is achieved and this is done in a way with less plant engineering complexity. In addition a highest possible reduction in the CO₂ emission should be achieved and ideally, if green electricity is available, a CO₂ emission-free process mode should be made possible.

This problem is solved by the embodiment of the process proposed in claim 1. Preferred embodiments are specified in the dependent claims. The process according to the invention is a process for separating butenes from a C4-hydrocarbon stream which contains at least butenes and butanes by extractive distillation with a solvent, wherein the process comprises the steps of:
a. at least partially evaporating the liquid C4-hydrocarbon stream in a feed evaporator, supplying the gaseous C4-hydrocarbon stream and supplying the liquid solvent above the C4-hydrocarbon stream to an absorber in which the C4-hydrocarbon stream and the solvent are contacted with one another to transfer predominantly butenes from the C4-hydrocarbon stream to the solvent, wherein the thus-laden solvent is collected in a liquid collector of the absorber and passed through an absorber evaporator and then passed into the bottom of the absorber below the liquid collector to outgas predominantly butanes from the laden solvent and wherein the laden solvent is subsequently passed to a desorber as bottoms stream;
b. supplying the laden solvent to the desorber, the bottom of which is at an elevated temperature and preferably a lower pressure relative to the bottom of the absorber and in which the butenes are separated from the solvent to obtain at the top of the desorber a stream enriched in butenes, wherein a solvent, at least partially freed of butenes, is collected in a liquid collector of the desorber and passed through a desorber evaporator and then passed into the bottom of the desorber below the liquid collector to outgas any butenes remaining in the solvent and wherein the solvent as bottoms stream of the desorber is subsequently recycled to the absorber as a heat source for a multi-stage high temperature heat pump;
characterized in that the heat of the solvent withdrawn as bottoms stream of the desorber is at least partially used for heat integration by employing the heat of the solvent in at least one respective heat exchanger for heat transfer in the heat pump, for evaporation in the absorber evaporator and for evaporation of the liquid C4-hydrocarbon stream; and in that
the heat for evaporation in the desorber evaporator is introduced via steam produced in the multi-stage high temperature heat pump, wherein the high temperature heat pump comprises at least a first container comprising a first heat exchanger, a second container comprising a second heat exchanger and at least one compressor, wherein
the first container contains a first operating medium to which heat from the solvent obtained as bottoms stream of the desorber is transferred in the first heat exchanger,
the second container contains a second operating medium to which heat from the first operating medium is transferred in the second heat exchanger.

One advantage of the present process is the relatively simple construction of the heat integration which nevertheless allows efficient energy recovery. The use of a heat pump additionally has the advantage that the process can be operated more independently, for example no heating medium in the desorber evaporator need be purchased. Such a heating medium must first of all be available in sufficient quantity at the site where the process is performed. Furthermore, energy recovery is optimized through the use of the heat pump. The waste heat of the solvent is raised to a particular temperature level via the heat pump so that it is directly employable at the desorber. The inventive configuration additionally makes it possible to save considerable amounts of heating medium. This in turn saves several kt per year of CO₂. Accordingly even a CO₂-free separation of n- and isobutane is performable if green electricity is employed. Furthermore, the separation of the butanes results in high energy savings and efficiency enhancements in an integrated system of two or more production plants because the inert butanes need not be entrained through the production plants. This especially applies if the butane-butene separation according to the invention is employed in the front portion of the integrated system.

The additional heat integration withdraws heat from the solvent originating from the desorber. The reason for this is not only that other streams or columns are to be heated but rather primarily the cooling of the solvent for the absorption. The absorption of the butenes (here: step a) is usually carried out at a lower temperature than the desorption (here: step b). If in the course of the heat integration sufficient heat is withdrawn from the solvent, i.e. it has a suitable temperature, the solvent may be passed directly into the absorber. However, it is also conceivable that despite the heat integration present the solvent does not yet have the correct temperature. In such a case the solvent may be passed through a residual cooler to be cooled to a suitable temperature after the heat integration and before entry into the absorber.

Heat is a process parameter. The heat supplied or removed corresponds to the change in internal energy minus the work done. The terms heat, heat transport and heat integration used in the present invention are always based on this definition.

The present process relates to the separation of butenes from butene-containing C4-hydrocarbon streams. These streams typically also contain alkanes (n-butane, isobutane) in addition to the butenes. In the context of the present invention-unless something else is being described-the term butanes is to be understood as meaning both n-butane and isobutane. The process according to the invention may therefore employ all C4-hydrocarbon streams which contain at least butenes and butanes provided that the amounts in which the butenes and/or butanes are present allow economic performance of the process. In a preferred embodiment of the present invention the employed C4-hydrocarbon stream consists essentially, i.e. to an extent of more than 98% by weight, preferably to an extent of more than 99% by weight, of butanes and butenes. The corresponding streams may also contain impurities or other hydrocarbons, such as 1,3-butadiene or C5-hydrocarbons, in small amounts.

The extraction process according to the invention employs a liquid solvent which dissolves primarily the butenes of the employed gaseous C4-hydrocarbon stream. Suitable solvents are aprotic solvents, for example N-methyl-2-pyrrolidone (NMP). The the process according to the invention is preferably performed with NMP as the solvent. In a further preferred embodiment of the present invention the solvent contains water, in particular in the range from 1% to 10% by weight, preferably from 4% to 9% by weight, in each case based on the total amount of solvent.

Absorbers that may be employed include in particular random-packed columns which comprise at least two random-packing beds. Such columns are known in principle to those skilled in the art. Preferably arranged above the first random-packed column is a backwashing zone comprising a plurality of theoretical trays to hold back the solvent entrained in the gas phase. Above the backwashing zone is the top of the absorber where a stream enriched in butanes relative to the employed C4-hydrocarbon stream is obtained. The liquid collector according to the invention would be arranged below the last random-packing bed, the bottom of the absorber being arranged below said collector. The precise construction of the absorber depends on various parameters and is variable in certain aspects.

The liquid solvent is supplied to the absorber spatially above the inlet for the C4-hydrocarbon stream. In a preferred embodiment the solvent is supplied to the absorber above the first random-packing bed and the C4-hydrocarbon stream is added to the absorber below the first random-packing bed. In the absorber the liquid solvent trickles downwards and is brought into contact with the (ascending) vaporous C4-hydrocarbon stream to transfer a portion of the C4-hydrocarbon stream containing predominantly butenes to the solvent to form a laden solvent. In step a the C4-hydrocarbon stream and the solvent are brought into contact with one another in particular in countercurrent. In a preferred embodiment of the present invention the portion of the C4-hydrocarbon stream transferred to the solvent comprises at least 70% by weight, particularly preferably at least 80% by weight, of butenes, based on the composition of the portion of the C 4-hydrocarbon stream transferred to the solvent. This has the result that in particular at least 80%, particularly preferably at least 90%, of the butenes present in the employed C4-hydrocarbon stream are transferred to the solvent.

The laden solvent flows downwards in the absorber and is collected in a suitable liquid collector, in particular a chimney tray. The temperature of the laden solvent is preferably between 40° C. and 90° C., particularly preferably between 45° C. and 65° C. The laden solvent is withdrawn from the liquid collector, passed through an absorber evaporator and then passed into the bottom of the absorber below the liquid collector to outgas predominantly butanes from the laden solvent. The absorber evaporator is preferably a once-through evaporator where the laden solvent is passed through the evaporator only once. This makes it possible to achieve the lowest possible temperatures, thus making it possible to prevent fouling. The driving temperature difference is additionally increased, thus allowing even more efficient energy utilization of the NMP stream. The absorber evaporator may also be configured as a multi-stage apparatus i.e. a plurality of heat exchangers/a plurality of evaporators belonging to the absorber evaporator may be present.

The solvent laden predominantly with butenes then remains in the bottom and is withdrawn therefrom and passed to the desorber as bottoms stream. The temperature in the bottoms stream of the absorber which is passed to the desorber is preferably between 70° C. and 130° C., particularly preferably between 85° C. and 120° C.

A stream enriched in butanes compared to the employed C4-hydrocarbon stream is then especially obtained at the top of the absorber. The overhead pressure in the absorber may be between 3 and 7 bar absolute, preferably between 4 and 6.5 bar absolute. The stream enriched in butanes may additionally contain water originating from the solvent. This water may be separated in a subsequent step. The stream enriched in butanes is withdrawn at the top of the absorber and subjected to a single-or multi-stage condensation to condense out a water-containing stream and a butane-containing product stream. These two streams may be separated from one another in a suitable apparatus, for example a spider. The water-containing stream separated from the butane-containing product stream may be passed to the absorber or to the desorber and/or partially discharged from the process depending on its composition.

The butane-containing product stream thus obtained from the condensation may still contain small amounts of water, in particular in an amount of up to 1500 ppmw, based on the total composition of the butane-containing product stream. In addition, the butane-containing product stream obtained from the condensation may further contain residual butenes, wherein the streams contain typically less than 20% by weight, preferably less than 15% by weight, particularly preferably less than 5% by weight, of butenes, based on the total composition of the butane-containing product stream.

Depending on the requirements for the obtained butane-containing product stream it may be necessary for the butane-containing product stream to be subjected after the condensation to a drying, preferably in a drying column, to separate the water still present. The butane-containing product stream preferably contains a maximum amount of water of 50 ppmw, preferably of 25 ppmw, after the drying. The water obtained during the drying may be recycled to the condensation in the absorber.

The solvent withdrawn at the bottom of the absorber and laden predominantly with butenes is supplied to the desorber. To this end the laden solvent may be passed to the desorber using a pump for example. Relative to the bottom of the absorber the bottom of the desorber is at an elevated temperature and preferably a lower pressure. The temperature in the bottom of the desorber is preferably between 120° C. and 200° C., more preferably between 130° C. and 195° C. The head pressure in the desorber may be between 1 and 6 bar absolute, preferably between 2 and 5 bar absolute. The elevated temperature and the preferably lower pressure relative to the absorber has the result that the butenes and any butanes still present are at least partially removed from the solvent. In a preferred embodiment the solvent at least partially freed of butenes contains up to 5000 ppmw of butenes, particularly preferably 100 to 900 ppmw of butenes. The solvent at least partially freed of butenes flows downwards in the desorber and is collected in the liquid collector of the desorber. From there the solvent at least partially freed of butenes is passed through a desorber evaporator and then passed into the bottom of the desorber below the liquid collector, in particular a chimney tray, to outgas any butenes remaining in the solvent. The desorber evaporator is preferably a once-through evaporator where the solvent at least partially freed of butenes is passed through the evaporator only once. This makes it possible to achieve the lowest possible temperatures, thus making it possible to prevent fouling. The desorber evaporator may also be configured as a multi-stage apparatus, i.e. a plurality of heat exchangers belonging to the desorber evaporator may be present. The solvent freed of butenes then remains in the bottom and is withdrawn therefrom, passed to the absorber as bottoms stream and reused there as solvent for the absorption of butenes.

Before being passed to the absorber the solvent freed of butenes may be partially or completely subjected to a regeneration to remove impurities, for example the abovementioned by-products present in the employed C4-hydrocarbon stream and/or formed from the butenes at the temperatures in the desorber such as oligomeric or polymeric compounds, from the solvent, preferably the NMP. The regeneration is preferably performed such that the solvent freed of butenes is passed into a container and evaporated at a pressure of less than 500 mbar absolute, more preferably of less than 200 mbar absolute and a temperature between 100° C. and 150° C. The container may have a column connected to it. Heavy boilers in particular are separated by the regeneration. If only a portion of the solvent freed of butenes is subjected to a regeneration the regeneration portion of the solvent is subsequently combined with the unregenerated solvent and recycled to the absorber.

A stream enriched in butenes compared to the employed C4-hydrocarbon stream is then especially obtained at the top of the desorber. This stream enriched in butenes may additionally contain water originating from the solvent. This water may be separated in a subsequent step. The stream enriched in butenes is withdrawn at the top of the desorber and subjected to a single-or multi-stage condensation to condense out a water-containing stream which may contain not only water but also residues of organics as well as a butene-containing product stream. These two streams may be separated from one another in a suitable apparatus, for example a spider. The water-containing stream separated from the butene-containing product stream may then be recycled to the desorber. Discharging the entirety or portions of the water-containing stream to remove the organics is also possible.

In a preferred embodiment of the present invention the condensation of the stream enriched in butenes withdrawn at the top of the desorber is configured as a two-stage condensation, wherein in a first stage a water-containing stream is condensed out and then recycled to the desorber and in the second stage the butene-containing product stream is condensed out. However, it may also be the case that any water present is also condensed out in the second stage. This residual water may be separated from the butene-containing product stream via a suitable apparatus, for example a spider.

The butene-containing product stream obtained from the condensation preferably contains less than 20% by weight, more preferably less than 16% by weight, of butanes based on the total composition of the butene-containing product stream. By contrast, the butene-containing product stream obtained from the condensation preferably has a butene content of at least 70% by weight, more preferably of at least 75% by weight, particularly preferably of at least 86% by weight, based on the total composition of the butene-containing product stream.

A characterizing feature of the present invention is the heat integration using the heat of the solvent on the way from the desorber to the absorber and of the hot condensate obtained in the desorber evaporator. The heat of the solvent, preferably of the NMP, withdrawn as a bottoms stream of the desorber is according to the invention used for heat integration by employing the heat of the solvent in at least one respective heat exchanger for heat transfer in the heat pump, for evaporation in the absorber evaporator and for evaporation of the liquid C4-hydrocarbon stream. According to the invention the solvent, preferably NMP, at least partially freed of butenes is collected in a liquid collector of the desorber and passed through a desorber evaporator to allow outgassing of any butenes remaining in the solvent. The heat for evaporation in the desorber evaporator may be introduced in a heat exchanger by heat transfer from a suitable heat transfer medium. The heat transfer medium is a steam produced by a heat pump integrated in the process and in the heat integration and may be in the pressure range of for example 5 to 30 bar, preferably in the range from 13 to 17 bar absolute. The condensation temperatures between 150° C. and 270° C. result from the reported pressures.

In the context of the present invention steam is produced in the multi-stage high temperature heat pump. It is in principle also possible to conceive of and employ a single-stage high temperature heat pump. The multi-stage high temperature heat pump comprises at least a first container comprising a first heat exchanger, a second container comprising a second heat exchanger and at least one compressor. It is possible to employ only a single multi-stage compressor which is capable of compressing various gases independently of one another. However, it is preferably also possible for the multi-stage high temperature heat pump to comprise at least two compressors, in each case at least one per stage.

The first container contains a first operating medium to which heat from the solvent obtained as bottoms stream of the desorber is transferred in the first heat exchanger. The first operating medium may in principle be a known heat transfer medium. However, it should be a medium which is not supercritical in the high temperature range (i.e. at temperatures >120° C.). In a preferred embodiment the first operating medium is selected from the group consisting of water, n-hexane, n-pentane, methanol and mixtures thereof. The first container may also be a kettle evaporator which comprises a heat exchanger integrated in a container.

The heat transfer from the solvent withdrawn as bottoms stream of the desorber to the first operating medium is effected in the first heat exchanger. The first heat exchanger may be connected to the bottom of the first container and the first operating medium may be recycled to the first container via the first heat exchanger. The first operating medium is heated and at least partially evaporated in the first heat exchanger. This also increases the pressure in the first container. The evaporated first operating medium can then be withdrawn at the container lid. The first operating medium is raised to a first temperature level by the heat transfer. The temperature of the first operating medium is preferably between 80° C. and 140° C. The pressure is preferably in the range from 2 to 8 bar absolute. The evaporated first operating medium is passed via the at least one compressor or via the first compressor to the second heat exchanger and from there recycled to the first container. During the recycling from the second heat exchanger the first operating medium may be passed through a further heat exchanger with which the evaporated first operating medium is further heated before passing through the at least one compressor or the first compressor.

The second container contains a second operating medium to which heat from the first operating medium is transferred in the second heat exchanger. The second operating medium may in principle be a known heat transfer medium. However, it should be a medium which is not supercritical in the high temperature range (i.e. at temperatures >120° C.). The second operating medium should preferably not be supercritical even at temperatures >150° C.. In a preferred embodiment the second operating medium is selected from the group consisting of water, n-hexane, n-pentane, methanol and mixtures thereof. The second container may also be a kettle evaporator which comprises a heat exchanger integrated in a container.

The heat transfer from the first operating medium to the second operating medium is carried out in the second heat exchanger. The second heat exchanger may be connected to the bottom of the second container and the second operating medium may be recycled to the second container via the second heat exchanger. The first operating medium is heated and at least partially evaporated in the second heat exchanger. This also increases the pressure in the second container. The evaporated first operating medium can then be withdrawn at the container lid. The heat transfer raises the first operating medium to a second temperature level, wherein the second temperature level is higher than the first temperature level. The temperature of the first operating medium is preferably between 80° C. and 140° C. The pressure is preferably in the range from 2 to 8 bar absolute. The evaporated second operating medium is passed as steam according to the invention via the at least one compressor or via the second compressor to the desorber evaporator and from there recycled to the second container. Before the evaporated operating medium arrives at the desorber evaporator fresh condensate can be injected to bring the temperature and the pressure to the desired level, preferably to cool the steam somewhat. During the recycling from the desorber evaporator the second operating medium may be passed through a further heat exchanger with which the evaporated second operating medium is further heated before passing through the at least one compressor or the second compressor.

The advantage of such an embodiment is obvious. The steam is intrinsically provided by the heat pump and need not be purchased. In addition, the utilization of the heat generated in the process is markedly greater than in other known processes. A markedly improved heat integration is thus achieved. A considerable amount of steam can also be saved because the hot condensate is better utilized in the present case.

In a preferred embodiment of the present invention the absorber comprises a dividing wall in the bottom by means of which the bottom is divided into two segments and the two-stage evaporation is performed such that the laden solvent collected in the liquid collector is passed through a first evaporator, preferably a once-through evaporator, and passed to the first segment and that the laden solvent from the first segment is passed through a second evaporator, preferably a forced circulation evaporator, and flashed into the second segment from which the bottoms stream is then withdrawn to the desorber. One advantage of this is that the configuration in the bottom of the absorber, i.e. the presence of a dividing wall and a two-stage evaporation, allows the height of the absorber to be reduced while achieving increased separation efficiency.

In a further preferred embodiment the desorber comprises a side evaporator. In such a case the heat transfer medium used for the side evaporator may be the mixed steam from the steam ejector while the desorber evaporator employs medium pressure steam as heating steam. The hot condensate from the desorber evaporator and the side evaporator are then passed to a condensate container as described hereinabove. The low pressure steam obtained there is then used in the steam ejector, whose mixing steam is employed in the side evaporator. The advantage of this variant is that the hot condensate obtained can be further decompressed to provide a greater amount of low pressure steam.

The presently described process may be employed in integrated chemical systems which comprise in particular an oligomerization and optionally a hydroformylation. It is possible for the separation of butenes by the process according to the invention to be employed at various points in the integrated plant. It is also possible for the separation of butenes according to the invention to be present at various points within an integrated chemical plant.

For example the process described here may be employed at the beginning of such an integrated system. The employed C4-hydrocarbon stream may then be in particular a crack C4, a raffinate 1, a raffinate 2 or a mixture thereof. If crack C4 and/or raffinate 2 are employed the separation process according to the invention may have arranged upstream of it a crack C4 hydrogenation in which butadiene is selectively hydrogenated or a butadiene separation in which butadiene is extractively removed with a solvent such as NMP or nitriles to reduce the content of butadiene. It is also possible to arrange a hydroisomerization downstream of an extractive butadiene separation and upstream of the separation according to the invention to facilitate the separation task in the process according to the invention since this converts 1-butene into 2-butene which is more readily absorbed by the solvent. The advantage of a separation of butanes is that the residence time in all reaction stages is higher because fewer inert butanes need be sent through the individual stages. An integration is desirable from an energetic standpoint especially downstream of the butadiene separation and upstream of the MTBE synthesis. As a result the inert i/n-butane can be separated from the integrated system early and need not pass through all subsequent distillation steps.

If the separation process is employed at the beginning of the integrated system the obtained product stream may be supplied to an MTBE synthesis which may then preferably be followed successively by a 1-butene separation, an oligomerization and one or more hydroformylations of the purified oligomers. A hydroformylation may be carried out not only with the product stream from the oligomerization, thus making it possible to produce for example INA (isononanol) from di-n-butenes after subsequent hydrogenation or ITDA (isotridecanal) from tributenes, but also with the unconverted butenes of the oligomerization, thus making it possible to produce 2-PH (2-propylheptanol) after subsequent aldol condensation followed by hydrogenation. The unconverted butenes from the oligomerization could optionally also be used to operate a further oligomerization instead of a hydroformylation. The conditions of the individual process steps are familiar to those skilled in the art. The individual process steps may comprise further steps, such as for example the separation of the products or the workup of the resulting stream, but these are not explicitly mentioned here. However, the process according to the invention may also be introduced at any other point of such an integrated system.

In one embodiment of the present invention the C4 hydrocarbon stream employed in the separation process according to the invention is withdrawn from an MTBE synthesis after separation of MTBE and the butene-containing product stream is subsequently supplied to a 1-butene separation, after which an oligomerization and one or more hydroformylations for subsequent production of 2-PH, ITDA and/or INA are successively carried out. The individual process steps may comprise further steps, such as for example the separation of the products or the workup of the resulting stream, but these are not explicitly mentioned here.

In a further embodiment of the present invention the C4-hydrocarbon stream employed in the separation process according to the invention is withdrawn from a 1-butene separation and the butene-containing product stream is subsequently supplied to an oligomerization, after which one or more hydroformylations for subsequent production of 2-PH, ITDA and/or INA are carried out. The individual process steps may comprise further steps, such as for example the separation of the products or the workup of the resulting stream, but these are not explicitly mentioned here.

In a further embodiment of the present invention the C4-hydrocarbon stream employed in the separation process according to the invention is withdrawn from an oligomerization and the butene-containing product stream is subsequently supplied to a hydroformylation for subsequent production of 2-PH. The individual process steps may comprise further steps, such as for example the separation of the products or the workup of the resulting stream, but these are not explicitly mentioned here.

In a further embodiment of the present invention the separation process according to the invention is employed at the end of the integrated system. In that case the employed C4-hydrocarbon stream is withdrawn from a 2-PH production downstream of the hydroformylation. The butene-containing product stream then obtained from the separation process according to the invention may in this case be recycled and employed at another suitable point in the integrated system, for example for 1-butene separation, for oligomerization or one or more hydroformylations. This makes it possible to enhance the efficiency of the overall integrated system since even after passing through the last process step in the integrated system up to 20% by weight of butenes may still be present.

Independently of the point in the integrated system where the separation process according to the invention is arranged the butane-containing product stream may be supplied for example to an adiabatic oligomerization, a hydrogenation of the butenes still present or an n/iso splitting of the butanes where n-butane and isobutane are separated from one another. The n/iso splitting may also be carried out after an adiabatic oligomerization. Another possibility would be inclusion of the butane-containing product stream upstream of the oligomerization in an above-described integrated system composed of MTBE synthesis, 1-butene separation, oligomerization and a hydroformylation.

In a particularly preferred embodiment of the present invention the energy required for n/iso splitting may be effected at least partially by heat integration with the first stage of a two-stage condensation at the top of the desorber. This has the advantage that the energy obtained in the condensation is utilized and not simply released to the environment as in the prior art.

The present invention is hereinbelow elucidated with reference to figures. The figures are for illustration but are not to be understood as limiting.

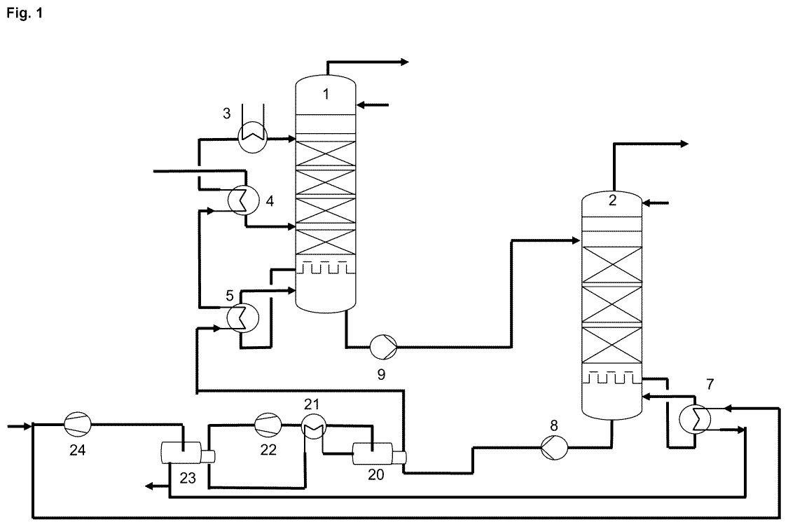

FIG. 1 shows the basic configuration of the present invention. The liquid C4-hydrocarbon stream is evaporated via a heat exchanger (4) and passed into the absorber (1). The solvent is-where necessary-brought to the desired temperature via a residual cooler and likewise passed into the absorber, wherein the inlet is spatially above the inlet for the C4 hydrocarbon stream, in the present case above the first random-packing bed. The stream enriched in butanes, which is withdrawn, is obtained at the top of the absorber (1). A possible condensation is not shown here, merely the recycling of a possible substream is indicated. The laden solvent is collected in the bottom of the absorber (1) as indicated by the chimney tray in the figure. There, at least a portion of the laden solvent is withdrawn and passed to the bottom of the absorber (1) via an absorber evaporator (5). The laden solvent is withdrawn from the bottom of the absorber (1) and using a pump (9) passed to the desorber (2) where the butenes present in the solvent are separated from the solvent. The stream enriched in butenes is obtained at the top of the desorber. This stream may be subjected to a single-or multi-stage condensation which is not shown in the figure. Only a possible recycle stream is indicated. The solvent at least partially freed of butenes is collected in the bottom of the desorber (2) as indicated by the chimney tray in the figure. There, at least a portion of the laden solvent is withdrawn and passed to the bottom of the desorber via a desorber evaporator (7). The solvent freed of butenes is then withdrawn from the bottom of the desorber (2) and using a pump (8) recycled to the absorber via the first container comprising the first heat exchanger (20), the absorber evaporator (5), the heat exchanger (4) for evaporating the C4-hydrocarbon stream and the residual cooler (3). In the first container comprising the first heat exchanger (20) at least a portion of the first operating medium is evaporated and passed via the further heat exchanger (21), where a further heating is carried out, and the first compressor (22) to the second container comprising the second heat exchanger (23). In the second container comprising the second heat exchanger (23) at least a portion of the second operating medium is evaporated and via the second compressor (24) passed to the desorber evaporator (7).

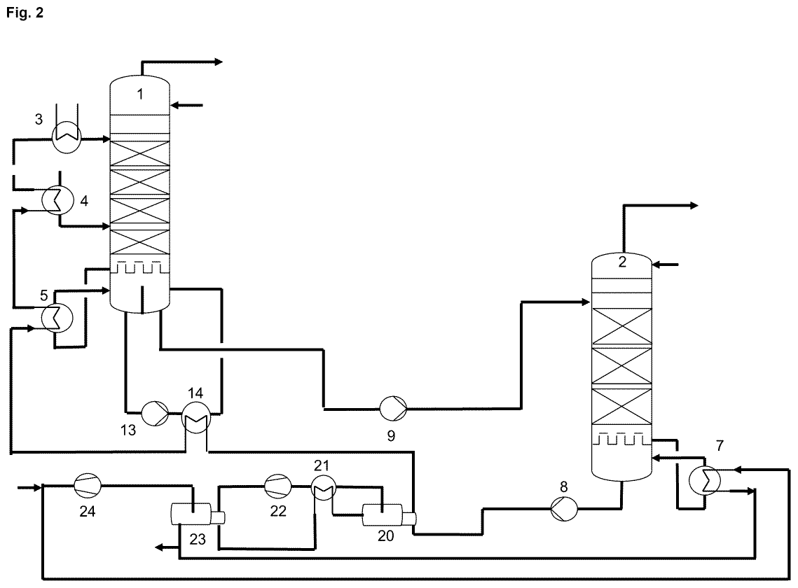

FIG. 2 shows a preferred embodiment of the present invention where a dividing wall is present in the bottom of the absorber (1). In this embodiment a portion of the laden solvent is withdrawn at the chimney tray of the absorber (1) and passed via an absorber evaporator (5) to the first segment in the bottom of the absorber (1) in which a dividing wall is present. The laden solvent is withdrawn from the first segment and with a pump (13) passed via a second evaporator (14) to the second segment in the bottom of the absorber. The laden solvent is withdrawn from the second segment in the bottom of the absorber (1) and using a pump (9) passed via the heat exchanger (6) for preheating the laden solvent to the desorber (2) where the butenes present in the solvent are separated from the solvent. In this preferred embodiment the heat exchanger transfers heat upon recycling of the solvent at least partially freed of butenes.

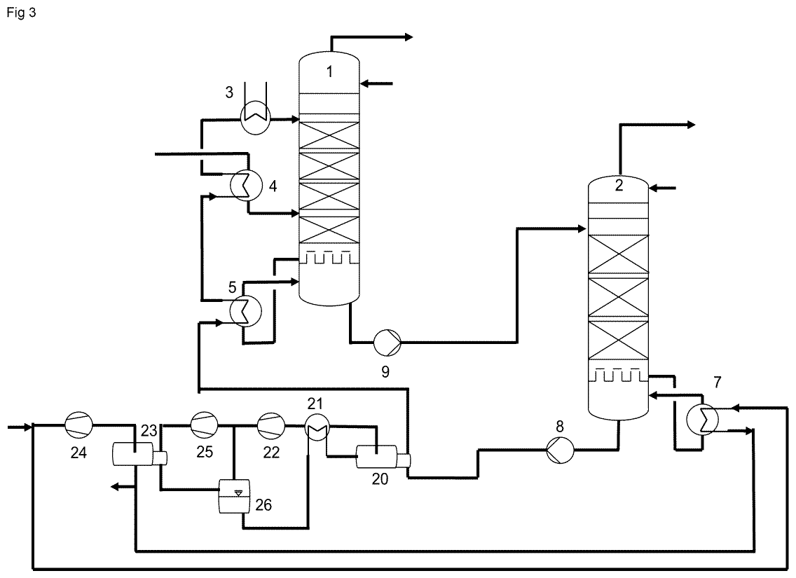

FIG. 3 shows a preferred embodiment of the present invention in which a flash vessel (26) for intermediate decompression (26) and an additional compressor (25) are present. Using the two compressors (22, 25) the first operating medium is passed to the second heat exchanger (23) and thus at least partially condensed. The first operating medium then passes into the flash vessel (26) and is separated therein. A portion passes via the additional heat exchanger (21) to the first container comprising the first heat exchanger (20). The other portion is passed between the two compressors (22 and 25), thus relieving the first compressor (22). It is thus no longer the entire operating medium that needs to be compressed from the low pressure level to the high pressure level but rather only a certain proportion thereof, thus entailing further energetic advantages.

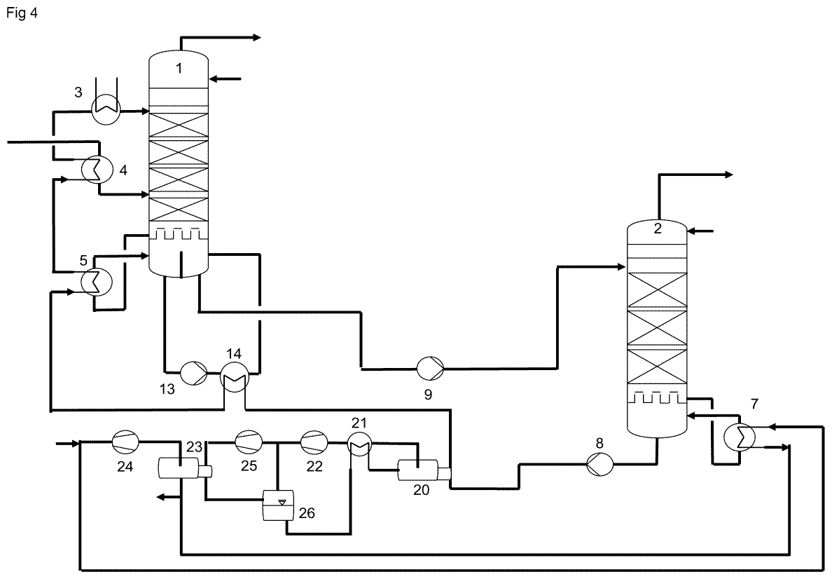

FIG. 4 shows an embodiment of the present invention where as per FIG. 2 a dividing wall is present in the absorber and where as per FIG. 3 the first operating medium is subjected to an intermediate decompression in the flash vessel (26).

The invention will now be elucidated with reference to a simulation. This embodiment is merely a preferred embodiment and is not to be understood as limiting.

### EXAMPLE

The butane-butene separation shown in FIG. 1 was simulated with Aspen Plus V10. A modified NRTL parameter set was used to describe interactions between the components. In the simulation 27 t/h of a hydrocarbon-containing feed were passed into a butane/butene separation. Said feed contains altogether 45% by weight of butanes (35% by weight n-butane and 10% by weight isobutane), 30% by weight of isobutene, 10% by weight of 1-butene, 10% by weight of trans-butene and 5% by weight of cis-butene.

Altogether 10 t/h of butane-containing tops product are withdrawn at the absorber. The butane-containing tops product of the absorber consists of 70% by weight of n-butane, 27% by weight of isobutane and 1% by weight of 1-butene and 2% by weight of isobutene. The solvent laden with the butene-containing product stream is withdrawn at the bottom. The butene-containing product stream contains 14% by weight of n-butane, 15% by weight of 1-butene, 47% by weight of isobutene, 8% by weight of cis-butene and 16% by weight of trans-butene. The established solvent/feed ratio of 13 accordingly allows a butene yield of 98% to be achieved. The large excess of solvent is then also used for the heat integration and as a heat source for the heat pump.

The solvent stream, here NMP, was passed from the bottom of the desorber (2) to a heat exchanger, a kettle evaporator (20), in which n-hexane is evaporated at 3.6 bar abs. The NMP is cooled from about 180° C. to 190° C. to about 120° C. to 125° C. and 13.4 MW are transferred. This forms an n-hexane recycle stream of slightly more than 240 t/h, which is superheated by 42 K at a gas superheater (21). The n-hexane stream is subsequently brought to a pressure level of 14.1 bar abs. with a multi-stage compressor (22). In a further heat exchanger, a further kettle evaporator (23), the operating medium n-hexane is condensed in the tubes. Water is evaporated at 7 bar abs. on the shell side. This steam is brought to a pressure level of 13.1 bar abs. with a further compressor (24). This forms about 35 t/h of steam which are used for evaporation in the desorber evaporator. Both operating media are recirculated.

Compared to conventional butane-butene separations where the heating media need to be purchased or provided the embodiment according to the invention ensures considerable savings potential of up to 35 t/h of steam. This amount is now produced internally by the heat pump.

1. Process for separating butenes from a C4-hydrocarbon stream which contains at least butenes and butanes by extractive distillation with a solvent, wherein the process comprises the steps of:
a. at least partially evaporating the liquid C4-hydrocarbon in a feed evaporator, supplying the gaseous C4-hydrocarbon stream and supplying the liquid solvent above the C4-hydrocarbon stream to an absorber in which the C4-hydrocarbon stream and the solvent are contacted with one another to transfer predominantly butenes from the C4-hydrocarbon stream to the solvent, wherein the thus-laden solvent is collected in a liquid collector of the absorber, passed through an absorber evaporator and then passed into the bottom of the absorber below the liquid collector to outgas predominantly butanes from the laden solvent and wherein the laden solvent is subsequently passed to a desorber as bottoms stream;
b. supplying the laden solvent to the desorber, the bottom of which is at an elevated temperature and preferably a lower pressure relative to the bottom of the absorber and in which the butenes are separated from the solvent to obtain at the top of the desorber a stream enriched in butenes, wherein a solvent, at least partially freed of butenes is collected in a liquid collector of the desorber and passed through a desorber evaporator and then passed into the bottom of the desorber below the liquid collector to outgas any butenes remaining in the solvent and wherein the solvent as bottoms stream of the desorber is subsequently recycled to the absorber as a heat source for a multi-stage high temperature heat pump;
characterized in that the heat of the solvent withdrawn as bottoms stream of the desorber is at least partially for heat integration by employing the heat of the solvent in at least one respective heat exchanger for heat transfer in the heat pump, for evaporation in the absorber evaporator and for evaporation of the liquid C4-hydrocarbon stream; and in that
the heat for evaporation in the desorber evaporator is introduced via steam produced in the multi-stage high temperature heat pump, wherein the high temperature heat pump comprises at least a first container comprising a first heat exchanger, a second container comprising a second heat exchanger and at least one compressor, wherein
the first container contains a first operating medium to which heat from the solvent obtained as bottoms stream of the desorber is transferred in the first heat exchanger,
the second container contains a second operating medium to which heat from the first operating medium is transferred in the second heat exchanger.

2. Process according to claim 1, wherein the heat pump comprises at least two compressors.

3. Process according to claim 1, wherein the first operating medium is selected from the group consisting of water, n-hexane, n-pentane, methanol and mixtures thereof.

4. Process according to claim 1, wherein the second operating medium is selected from the group consisting of water, n-hexane, n-pentane, methanol and mixtures thereof.

5. Process according to claim 1, wherein the first operating medium and the second operating medium are not identical.

6. Process according to claim 1, wherein the first operating medium is at least partially evaporated by the heat introduced during the heat transfer.

7. Process according to claim 6, wherein the evaporated operating medium is passed via the at least one compressor to the second heat exchanger and from there recycled to the first container, preferably via a further third heat exchanger with which the evaporated first operating medium is further heated before passing through the at least one compressor.

8. Process according to claim 7, wherein the second operating medium is at least partially evaporated by the heat introduced during the heat transfer.

9. Process according to claim 8, wherein the second operating medium is passed via the at least one compressor to the desorber evaporator and from there recycled to the second container in at least partially condensed form, preferably via a further fourth heat exchanger with which the second operating medium is further heated before passing through the second compressor.

10. Process according to claim 1, wherein the employed solvent is NMP.

11. Process according to claim 10, wherein the solvent/the NMP contains water and the water content is between 1% and 10% by weight, preferably between 4% and 9% by weight.

12. Process according to claim 1, wherein a stream enriched in butanes compared to the employed C4-hydrocarbon stream is obtained at the top of the absorber.

13. Process according to claim 1, wherein the temperature in the bottoms stream of the absorber which is passed to the desorber is between 70° C. and 130° C., preferably 85° C. to 120° C.

14. Process according to claim 1, wherein the temperature in the bottom of the desorber is between 120° C. and 200° C., preferably 130° C. and 195° C.
